(19) United States
(12) Patent Application Publication
(10) Pub. No.: US 2015/0305987 A1
(43) Pub. Date: Oct. 29, 2015

(54) POLYMERIZABLE MIXTURE COMPOSITION, USE OF SAID MIXTURE COMPOSITION, AND A DENTAL PROSTHETIC

(71) Applicant: MERZ DENTAL GMBH, Lütjenburg (DE)

(72) Inventors: Sebastian Pflesser, Kiel (DE); Stefan Seifert, Lütjenberg (DE); Ralf Zimehl, Wendtorf (DE)

(73) Assignee: Merz Dental Gmbh, Lütjenburg (DE)

(21) Appl. No.: 14/413,398

(22) PCT Filed: Jul. 8, 2013
(86) PCT No.: PCT/EP2013/002010

§ 371 Date: May 15, 2015

(30) Foreign Application Priority Data
Jul. 6, 2012 (DE) 10 2012 013 514.9

(51) Int. Cl. A61K 6/093; A61K 6/087

(52) U.S. Cl. CPC A61K 6/093; A61K 6/087

(57) ABSTRACT

The invention describes a polymerisable mixture composition containing a liquid or semi-solid component A with at least one monomer portion and one, preferably powdery, solid component B on a polymethyl methacrylate (PMMA) base with a filler and/or additives, the liquid or semi-solid component A additionally having at least one oligomeric or polymeric compound modifying the monomer portion and which is miscible with the monomer portion. Furthermore, use of the mixture composition and a moulded part, for example a dental prosthesis, produced from the latter, is specified.

The present invention relates to a polymerisable mixture composition containing a liquid or semi-solid component A with at least one monomer portion and one solid component B on a polymethyl methacrylate (PMMA) base with a filler and/or additives, the use of the mixture composition and a dental implant or prosthesis, produced from the mixture composition.

Conventional plastics for moulded parts, in particular commercially available prosthesis base plastics, with a polymethyl methacrylate base in the dental domain, only have a very low energy of rupture and average fracture toughness due to the physical properties of polymethyl methacrylate (PMMA).

PMMA and its commercially available copolymers have a very high degree of hardness and brittleness or fragility, and when stressed this initially means that a relatively high force must be overcome in order to bring about material fracture by means of crack propagation. This leads to an average fracture toughness value which specifies how much energy has to be expended for crack propagation. However, if this energy is overcome in the first place, the crack instantly continues through all of the material and shatters it. For example, a dental prosthesis would then no longer be useable and would have to be repaired wherever possible.

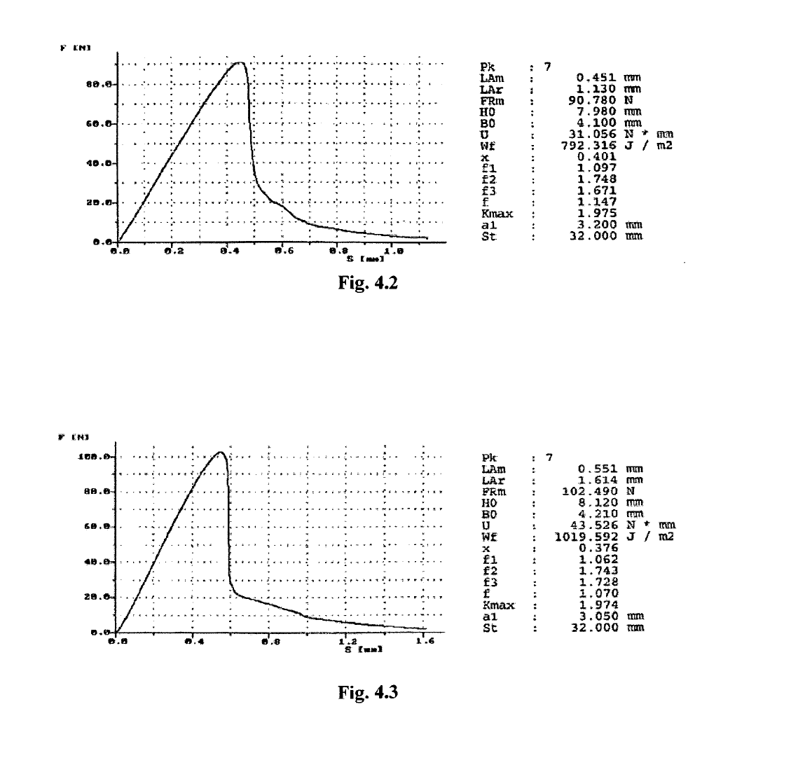

On the basis of this fracture behaviour of PMMA, a very low value is given for energy of rupture. This specifies how much energy is required per unit of area in order to shatter a pre-damaged, standardised test specimen. Since the energy expended in a chip fracture falls abruptly to 0, this work (energy of rupture, W_{f}) is very small—see the graphical illustration in FIG. 4.1.

In a fracture-tough material, however, the crack propagation starts at an energy maximum, defined as fracture toughness, (Kₘₐₓ), but this crack then only moves slowly through the test specimen because the material has significantly increased toughness due to a modification and so is able to dissipate the forces acting at the tip of the crack. As a result, a fracture-tough material can tolerate significantly more force/area before the crack reaches the opposite side of the test specimen/moulded part and possibly even shatters it; see the graphic illustration in FIGS. 4.2 and 4.3 where the scale of the graphs should be noted.

In order to produce individual moulded parts such as e.g. dental prostheses, different processes with the corresponding materials are available on the market. The casting process with autopolymerising PMMA-based plastics is gaining an ever-increasing market share. This is generally a 2-component system which on the one hand consists of a liquid component, predominantly consisting of a monofunctional polymerisable monomer, in particular methyl methacrylate, a cross-linking agent, in particular difunctional alkyl dimethacrylates, e.g. ethylene glycol dimethacrylate or 1,4-butanediol dimethacrylate, optionally additives for regulating the molar mass of the resulting polymers, e.g. thiols, and low concentrations of a corresponding initiator system, e.g. peroxide/amine systems or systems based on barbituric acid derivatives, stabilsers for storage, e.g. hydroquinone monomethyl ethers and/or colour- or microbially effective compounds.

On the other hand, a solid, preferably powdery component is available which predominantly comprises polymethyl methacrylate and its copolymers of any compositions and in any mixture ratios, in particular in bead form. Optionally, fillers, e.g. silicates or apatites, dyes, e.g. azo condensation products, and pigments, predominantly iron and titanium oxides, and modifiers, e.g. for adjusting an X-ray opacity, or microbiologically effective materials and additional initiator components, are additionally added to the solid (second) component.

The two aforementioned components are generally mixed in a fixed ratio to one another so that a pourable mixture is produced. Due to the fact that PMMA and most of its non-cross-linked copolymers dissolves, or at least can be soaked, in its monomer or MMA, the consistency of the mixture continuously increases as time passes and is poured, for example, into an appropriately prepared dental mould at once. Within the latter the mixture becomes more and more pasty, the initiator components starting the polymerisation promptly.

In this connection, publications EP 1702633 and DE 102005012825 disclose a fracture-tough autopolymerising “high impact material” (“PalaXpress Ultra”, Heraeus Kuizer). In these publications the use of elastically modified bead polymerisates in autopolymerising 2-component dental prosthesis base materials are described in order to achieve the values relating to fracture toughness and energy of rupture required according to the DIN EN ISO standard. The elastic modification of the bead polymerisates takes place here by means of core/shell particles of any type, e.g. by means of classic core/sheath particles (with an elastic core), multi-core particles and a number of core/sheath particles in a solid matrix or by means of inter-penetrating networks from the elastic and solid phase within the bead polymerisates.

However, it is a disadvantage here that only the polymer component is modified. After mixing and polymerising with the monomer component a material structure composed of circular, fracture-toughness-modified polymer beads is produced (see paras. 6.1 and 6.2) which are connected by the polmerised monomer matrix. Since the monomer matrix has not been modified, however, the latter still has a high degree of brittleness and fragility, and so low fracture toughness. In addition, in this type of polymer blend it is not guaranteed that the modified particles will be distributed homogeneously. In unfavourable cases, such intrinsic inhomogeneities lead to undesired predetermined breaking points.

It is therefore the object of the present invention to specify a pourable, autopolymerising mixture composition which has significantly increased energy of rupture and fracture toughness in comparison to conventional PMMA materials, preferably corresponding to the requirements and definitions according to product standard DIN EN ISO 20795-1:2008 and avoids the aforementioned disadvantages by means of inherent material and composition properties. Furthermore, it is the object of the invention to provide a suitable use for one such composition and a corresponding mould.

This object is achieved by the features of independent and additional independent claims 1, 10 and 13.

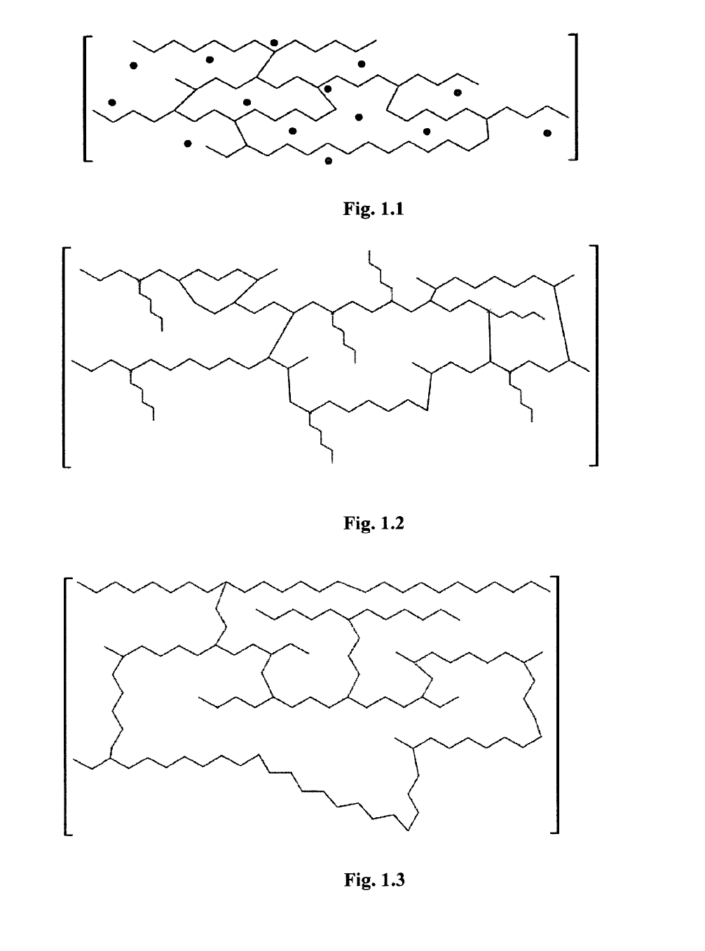

One essential aspect of the invention is the preparation of a polymerisable mixture composition containing a liquid or semi-solid component A with at least one monomer portion and one, preferably powdery, solid component B on a polymethyl methacrylate (PMMA) base with a filler and/or additives, the liquid or semi-solid component A additionally having at least one oligomeric or polymeric compound that modifies the monomer portion and which is miscible with the monomer portion. In other words, it is not the solid polymer component that is modified here as regards the shatter properties of the mould obtained after the polymerisation, but rather, advantageously, the liquid monomer portion in component A. Added to the monomer mixture is, for example, a linear poly(organo)siloxane which mixes with the liquid component A and does not bring about any phase separation at a later stage either, and can be polymerised into the system. This can be achieved, on the one hand, by the physical route, i.e. by depositing so-called outer modifiers, see for example FIG. 1.1, or chemically by the formation of covalent bonds and the development of copolymers as so-called inner modifiers, see FIGS. 1.2 and 1.3.

The powdery solid component B can be at least wetted here by the liquid or semi-solid, i.e. viscous or optionally pasty component A containing the monomer, can preferably be dispersed in the latter, particularly preferably can be soaked, and in the ideal case, particularly preferably, can be totally dissolved in the liquid or semi-solid pasty component A.

Preferably, the solid component B and/or the liquid or semi-solid component A contains initiator components which trigger the polymerisation single-handedly upon mixing.

Preferably, the portion of oligomeric or polymeric compound in the liquid or semi-solid component A comes within a range of between 0.1 and 50% by weight or between 0.1 and 30% by weight, preferably between 0.5 and 25% by weight, and particularly preferably between 1 and 20% by weight.

Preferably, at least one of the oligomeric or polymeric compounds is a polyorganosiloxane.

Preferably, the polymer compound or the at least one polyorganosiloxane is selected from the group consisting of polydimethylsiloxanes (PDMS), polydiphenylsiloxanes (PDPS), polymethylphenylsiloxanes (PMPS) and/or mixtures of the latter.

The polyorganosiloxane is preferably homogeneously miscible with the monomer portion of the liquid or semi-solid component A and/or preferably has a linear chain structure.

Here the average number (n) of linear polyorganosiloxane groups preferably comes within a range of between 2 and 500 or between 2 and 200, preferably between 5 and 200 or between 5 and 150, particularly preferably in the range of between 7 and 75 or between 7 and 40.

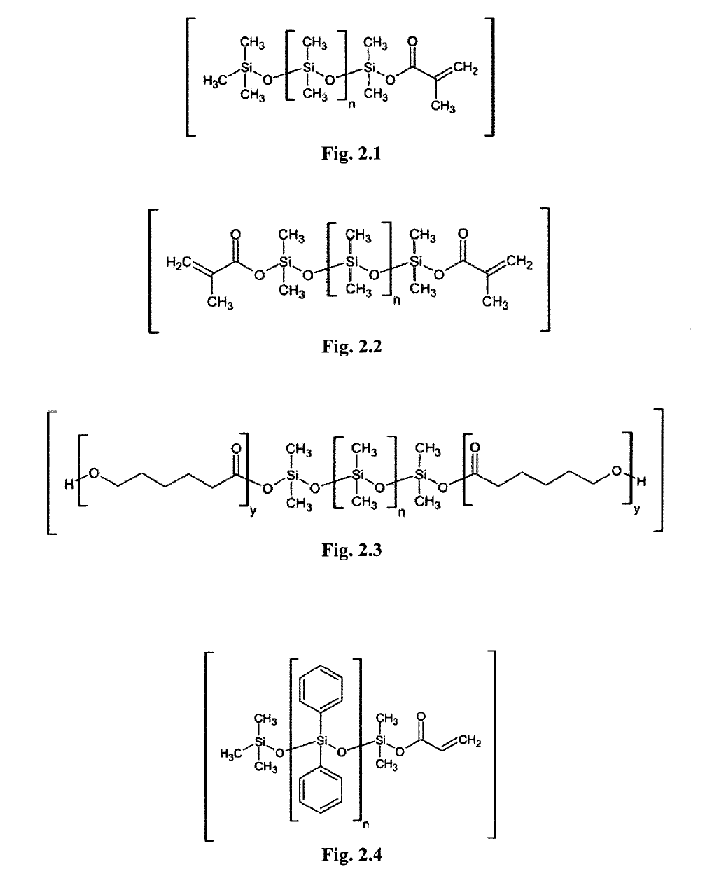

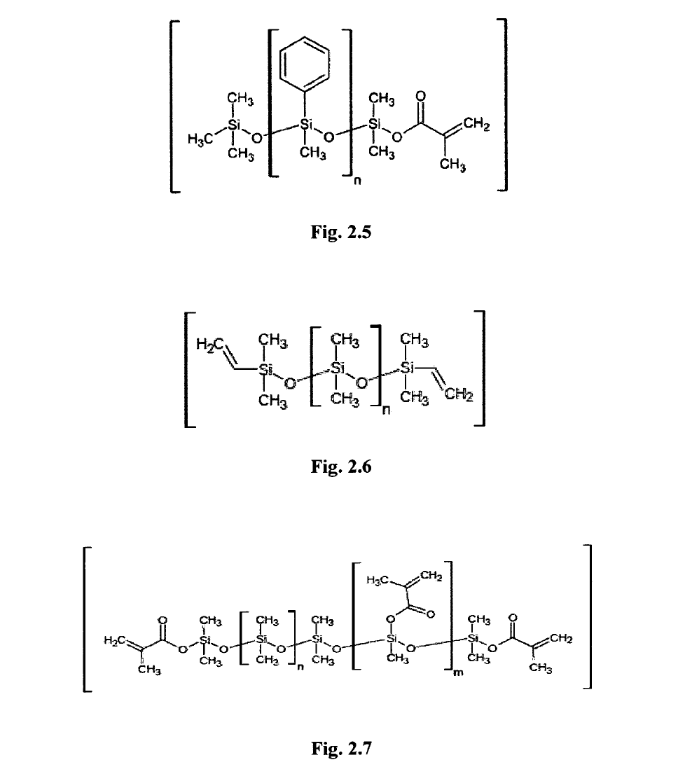

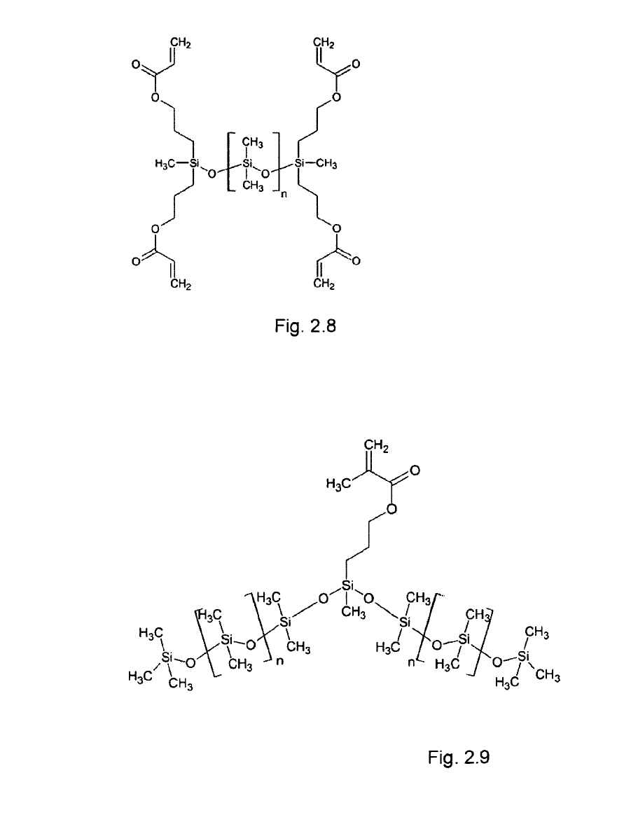

The polyorganosiloxane chain structure is also preferably modified, in particular the latter is modified chemically by means of substituents. This modification preferably involves polymers that tolerate PMMA systems, i.e. are miscible and/or can be converted with the latter, e.g. (poly)caprolactones, see FIG. 2.3, particularly preferably unsaturated groups such as e.g. vinyl (see FIG. 2.6), allyl and (meth)acryl groups (see FIGS. 2.2, 2.8 and 2.9) and very particularly preferably a (one-sided) chain termination with a (meth)acryl group according to FIG. 2.1, FIG. 2.4 and FIG. 2.5.

In one preferred embodiment the polymerised mixture composition or the plastic mould has a fracture toughness (Kₘₐₓ)≧1.9 kJm^{1/2} and an energy of rupture ≧900 J/m² or the plastic mould has both an increased fracture toughness (Kₘₐₓ) and an increased energy of rupture (W_{f}) in comparison to an unmodified and polymerised mixture composition.

In one use according to the invention of the aforementioned polymerisable mixture composition for producing a plastic mould the polymerised mixture composition or the plastic mould has a fracture toughness (Kₘₐₓ)≧1.9 kJ/m^{1/2} and an energy of rupture 900 J/m² according to DIN EN ISO 20795-1:2008.

The plastic mould produced in this way is preferably used as a dental prosthesis body and in one embodiment satisfies the minimum requirements according to ISO for high impact plastics.

Preferably the polymerisable mixture composition is used here as a repair material for this type of mould or the like. In particular, this also includes use as repair material for hot polymerisates.

Furthermore, according to the invention a dental prosthesis is provided which is produced from one of the polymerisable mixture compositions defined above.

The following examples and figures are intended to illustrate the invention and its advantages. These show as follows:

FIG. 1.1 a PMMA network according to the invention with an outer modifier (e.g. W35);

FIG. 1.2 a PMMA network according to the invention with an inner modifier (e.g. fluid MA-15M);

FIG. 1.3 a PMMA network according to the invention with a cross-linking modifier (e.g. fluid MA-40D);

FIG. 2 examples of poly(organo)siloxanes according to the invention;

FIG. 2.1 mono-MA-functional PDMS (e.g. fluid MA-15M);

FIG. 2.2 di-MA-functional PDMS (e.g. fluid MA-40D);

FIG. 2.3 polycaprolactone-modified PDMS (e.g. W35);

FIG. 2.4 mono-AC-functional PDPS;

FIG. 2.5 mono-MA-functional PMPS:

FIG. 2.6 di-vinyl-functional PDMS;

FIG. 2.7 comb-shaped multi-MA-functional PDMS;

FIG. 2.8 terminally multiacrylated PDMS;

FIG. 2.9 centrally methacrylated PDMS;

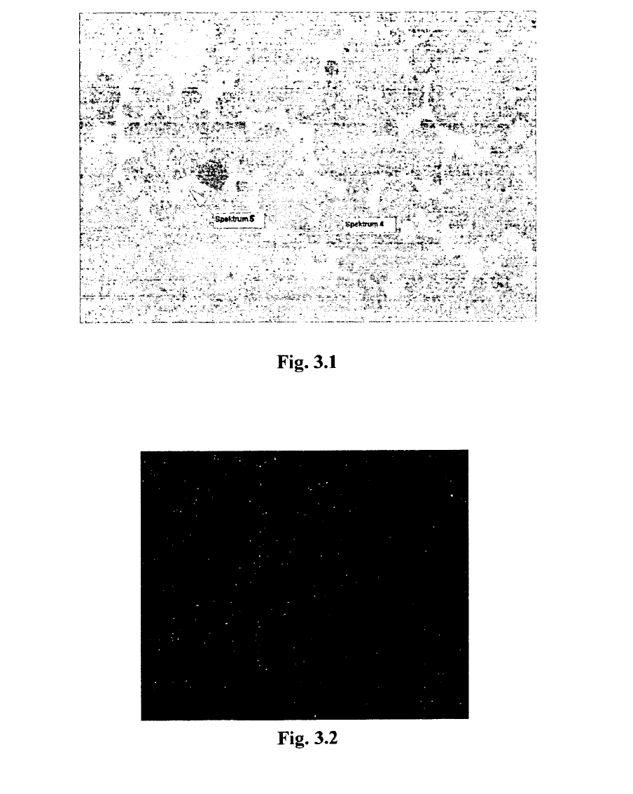

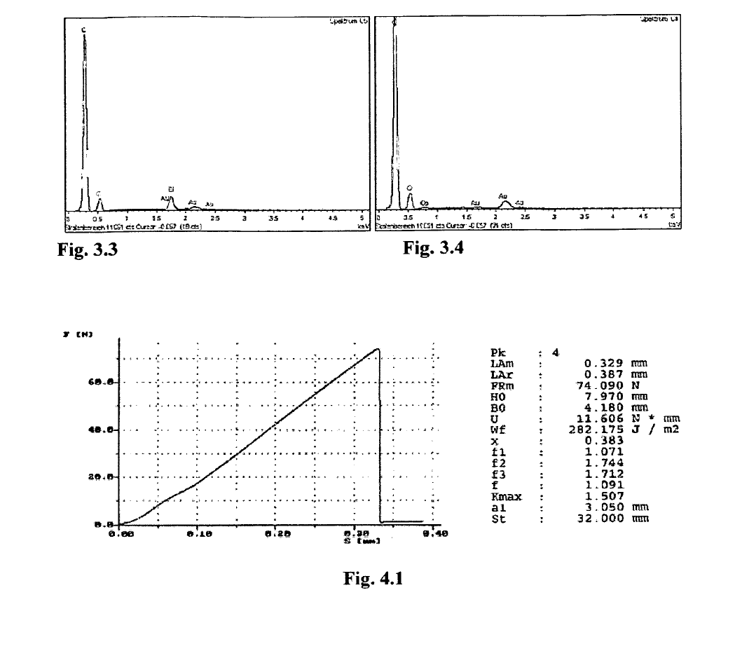

FIG. 3.1 SEM picture of a polymer structure of a dental plastic with two EDX measuring points (see FIG. 3.3 and FIG. 3.4);

FIG. 3.2 silicon-selective element mapping (EDX) in the SEM of a moulded part according to the invention;

FIG. 3.3 EDX spectrum of the matrix surrounding the beads;

FIG. 3.4 EDX spectrum of the bead polymerisate;

FIG. 4.1 force diagram of a polymer moulded part from the prior art (standard mixture);

FIG. 4.2 force diagram of a polymer moulded part according to the invention (Example 4, see below);

FIG. 4.3 force diagram of a polymer moulded part according to the invention (Example 2, see below);

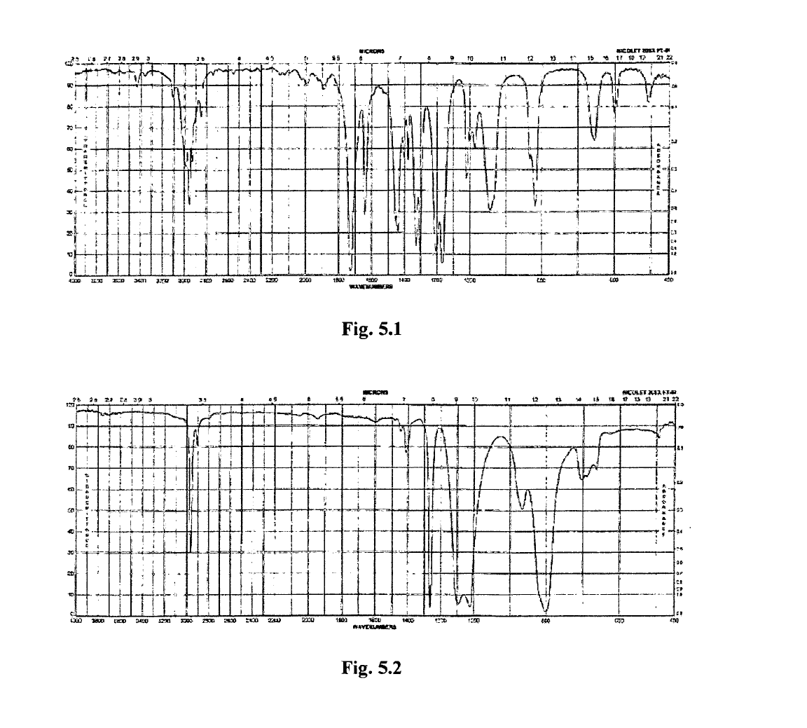

FIG. 5.1 IR spectrum of methyl methacrylate (Sigma-Aldrich);

FIG. 5.2 IR spectrum of polydimethylsiloxane (Sigma-Aldrich);

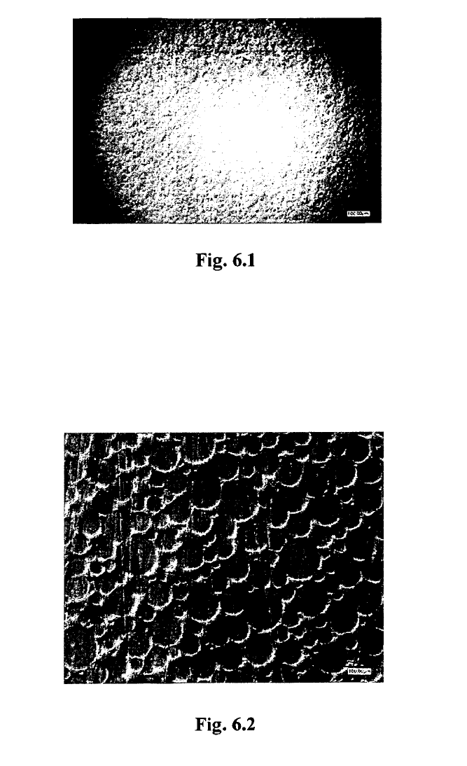

FIG. 6.1 structure of an autopolymerised PMMA plastic (100× enlargement) and

FIG. 6.2 structure of an autopolymerised PMMA plastic (600× enlargement.

### EXAMPLES

### Composition of a General Polymer Component

97 g MW134 (polymethyl methacrylate copolymer, made by the Evonik company) and 3 g 1-benzyl-5-phenyl-barbituric acid.

Composition of a monomer mixture (standard mixture) from the prior art: 95.6 methyl methacrylate, 4 g 1,4-butanediol dimethacrylate, 0.2 g dilauryl dimethyl ammonium chloride and 0.2 g copper naphthenate concentrate are combined and, after the chloride has dissolved, form a crystal clear, homogeneous solution.

### Example 1 of a Composition According to the Invention of a (Monomer) Component A

93.6 g methyl methacrylate, 4 g 1,4-butanediol dimethacrylate, 2 g fluid MA-15M, 0.2 g dilauryl dimethyl ammonium chloride and 0.2 g copper naphthenate concentrate are combined and, after the chloride has dissolved, form a crystal clear homogeneous solution. Fluid MA-15M is a thin liquid, clear, linear polydimethylsiloxane with an average chain length of n=15 and a one-sided terminal methacryl group (see FIG. 2.1).

### Example 2 of a Composition According to the Invention of a (Monomer) Component A

80.6 g methyl methacrylate, 4 g 1,4-butanediol dimethacrylate, 15 g fluid MA-15M, 0.2 g dilauryl dimethyl ammonium chloride and 0.2 g copper naphthenate concentrate are combined, and after the chloride has dissolved, form a crystal clear, homogeneous solution.

### Example 3 of a Composition According to the Invention of a (Monomer) Component A

90.6 methyl methacrylate, 4 g 1,4-butanediol dimethacrylate, 5 g W35, 0.2 g dilauryl dimethyl ammonium chloride and 0.2 g copper naphthenate concentrate are combined and, after the chloride and the W35 have dissolved, form a crystal clear, homogeneous solution. W35 is a linear polydimethylsiloxane with an average chain length n=40, the chain ends of which have been modified with a polycaprolactone (average chain length y=15 per side). It has a waxy structure, but dissolves well in MMA and the co-monomers of the latter (see FIG. 2.3).

### Example 4 of a Composition According to the Invention of a (Monomer) Component A

85.4 methyl methacrylate, 4 g butanediol dimethacrylate, 10 g fluid MA-40D, 0.2 g dilauryl dimethyl ammonium chloride and 0.2 g copper naphthenate concentrate are combined and, after the chloride has dissolved, form a crystal clear, homogeneous solution. Fluid MA-40D is a thin liquid, clear, linear polydimethylsiloxane with an average chain length of n=40, and an end group modification on both sides, each with a methacryl group (see FIG. 2.2).

### Example 5 of a Composition According to the Invention of a (Monomer) Component A

91.6 methyl methacrylate, 4 g 1,4-butanediol dimethacrylate, 4 g MD-M-2520 Mu, 0.2 g dilauryl dimethyl ammonium chloride, 0.2 g copper naphthenate concentrate are combined and, after the chloride has dissolved, form a crystal clear, homogeneous solution. MD-M-2520Mu is a liquid, clear, linear PDMS with an average chain length of n=35 and a multiply acrylated chain termination on both sides (see FIG. 2.8).

### Example 6

### Polymerisation and Testing of the Compositions from Examples 1-4 and of the Standard Mixture

20 g of polymer component B are mixed with 14 ml of a monomer component A, poured into a rectangular mould and polymerised at 2 bar and 50° C. over a period of 15 minutes in a dental pressure pot. The sheet of the standard mixture is almost crystal clear, whereas the modified sheets have a clear but homogeneous opacity, the latter being of such an intensity that the materials can be adjusted to the commercially available dental colours.

The physical values relating to energy of rupture (W_{f}) and fracture toughness (K) as well as flexural strength (FS) and flexural modulus (FM) are tested according to DIN EN ISO 20795-1:2008, see Table 1.

TABLE 1

| Standard | FM = | FS = | W_{f} = | K = |
|---|---|---|---|---|
| mixture | 2571 ± 54 | 83.8 ± 2.3 | 295 ± 16 | 1.53 ± 0.09 |
| 1^{st} | FM = | FS = | W_{f} = | K = |
| mixture A | 2511 ± 70 | 79.9 ± 2.7 | 848 ± 28 | 2.44 ± 0.06 |
| 2^{nd} | FM = | FS = | W_{f} = | K = |
| mixture A | 1936 ± 53 | 51.7 ± 2.6 | 941 ± 88 | 1.92 ± 0.09 |
| 3^{rd} | FM = | FS = | W_{f} = | K = |
| mixture A | 2481 ± 100 | 114.6 ± 1.3 | 592 ± 70 | 1.87 ± 0.06 |
| 4^{th} | FM = | FS = | W_{f} = | K = |
| mixture A | 2198 ± 94 | 74.7 ± 12.3 | 710 ± 119 | 1.78 ± 0.11 |
| 5^{th} | | | W_{f} = | K = |
| mixture A | | | 696 ± 22 | 1.85 ± 0.10 |

It is shown that additions of modified polyorganosiloxanes to the monomer mixture give a significant increase in relation to the physical values required by the standard with regard to the overall energy of rupture and the fracture toughness, and mixture A from Example 2 according to the invention particularly advantageously fulfils the limit values for material properties to be highlighted with W_{f}≧900 and K≧1.9, and mixture 1 according to Example 1 more or less achieves these requirements by approximately maintaining FM and FS.

The main portion of monomer component A is with >80% methyl methacrylate and so guarantees compatibility/solubility with or of polymer component B and, for example, to the artificial teeth incorporated during processing or the contact points with materials that are already polymerised when used as repair material. During use, and in particular during the production process, it is significantly easier to mix liquid or semi-solid components than to dissolve solids, and this constitutes a significant improvement in comparison to the use of conventional materials.

By modifying the monomer component with liquid materials, homogeneous distribution of the additives is guaranteed, see FIG. 3.2, whereas with conventional solid distribution in component B this does not occur perfectly or totally homogenously.

Due to the homogeneous distribution of the siloxanes in monomer component A, these are also particularly advantageously distributed homogeneously within the workpiece at a later point and provide consistent physical properties—something which is not guaranteed with solids due to the possible formation of agglomerates or sedimentation of the powdery materials due to different grain sizes and/or densities.

By adding poly(organo)siloxanes the later workpiece is moreover adjusted to be more water-repellent in its entirety, and so reduces water absorption and water solubility. The lower water absorption advantageously leads to fewer bacteria or less plaque being able to accumulate on the workpiece, particularly advantageously when processing the workpiece to form the later dental prosthesis. Furthermore, by using polysiloxanes in the composition the material surface can be better polished. This leads advantageously to a better surface quality and better resistance to deposits.

On the basis of the unsaturated groups which are preferably present, in particular (meth)acryl group(s), the polysiloxanes are integrated chemically into the polymer network and so this advantageously prevents subsequent diffusing out.

Due to the significant improvement of the required physical properties of the moulded part and the simplified manageability of the composition by means of the exclusive modification of the monomer component, a new class of base material is moreover provided for the production of prosthesis.

Moreover, by adding polysiloxanes to the monomer component, the percentage portion of polymerisable groups is reduced, and so the overall volumetric shrinkage of the workpiece caused by polymerisation is advantageously reduced.

If the poly(organo)siloxanes are modified with phenyl groups, their refractive index is additionally matched to that of PMMA so that the transparency significantly increases with respect to conventional poly(organo)siloxanes and so an additional advantage is offered with respect to the standard modification (see FIGS. 2.4 and 2.5).

If poly(organo)siloxanes are used which additionally carry other unsaturated groups along the siloxane chain (see FIG. 2.6) and so have a comb-like structure, compatibility with the system is significantly improved and higher concentrations can be used in order to optimise the physical values.

The IR spectra according to FIG. 5.1 and FIG. 5.2 show significant differences with respect to the spectra of conventional materials, e.g. 1070: antisymmetrical stretching vibration Si—O—Si, 800 and 840: mono and dimethylsiloxane groups; see Infrared spectra and structure of thin polydimethylsiloxane films, E. A. Romaneko and B. V. Tkachuk, and so the addition of poly(organo)siloxanes in the monomer component can be used by means of IR spectroscopy for the chemical identification.

In the polymerised material, poly(organo)siloxanes can be identified by SEM-EDX pictures, see FIG. 3.1 to FIG. 3.4, since due to the use of modifiers in the monomer component, the latter can be detected easily in the intermediate regions surrounding the beads.

All of the features specified in the present documents are claimed as essential to the invention.

1. A polymerisable mixture composition containing a liquid or semi-solid component A with at least one monomer portion and one solid component B on a polymethyl methacrylate (PMMA) base with a filler and/or additives, the liquid or semi-solid component A additionally having at least one oligomeric or polymeric compound modifying the monomer portion and which is miscible with the monomer portion.

2. The polymerisable mixture composition according to claim 1, characterised in that the solid component B and the liquid or semi-solid component A contain initiator components.

3. The polymerisable mixture composition according to claim 1 or 2, characterised in that the portion of oligomeric or polymeric compound in the liquid or semi-solid component A comes within a range of between 0.1 and 50% by weight, preferably between 0.1 and 30% by weight, preferably between 0.5 and 25% by weight, and particularly preferably between 1 and 20% by weight.

4. The polymerisable mixture composition according to any of claims 1 to 3, characterised in that at least one oligomeric or polymeric compound is a polyorganosiloxane.

5. The polymerisable mixture composition according to any of claims 1 to 4, characterised in that the polymer compound or the at least one polyorganosiloxane is selected from the group consisting of polydimethylsiloxanes (PDMS), polydiphenylsiloxanes (PDPS), polymethylphenylsiloxanes (PMPS) and/or mixtures of the latter.

6. The polymerisable mixture composition according to claim 4 or 5, characterised in that polyorganosiloxane is homogeneously miscible with the monomer portion of the liquid or semi-solid component A and/or has a preferably linear chain structure.

7. The polymerisable mixture composition according to claim 6, characterised in that the average number (n) of linear polyorganosiloxane groups comes within a range of between 2 and 500, preferably between 2 and 200, preferably between 5 and 200, preferably between 5 and 150, particularly preferably in the range of between 7 and 75, preferably between 7 and 50.

8. The polymerisable mixture composition according to either of claim 6 or 7, characterised in that the polyorganosiloxane chain structure is chemically modified.

9. The polymerisable mixture composition according to claim 8, characterised in that the polyorganosiloxane chain structure is chemically modified by means of substituents, preferably by means of polycaprolactones, particularly preferably unsaturated groups, preferably (meth)acryl groups.

10. The polymerisable mixture composition according to any of claims 1-9, characterised in that the polymerised mixture composition or the plastic mould has a fracture toughness (Kₘₐₓ)≧1.9 kJm^{1/2} and an energy of rupture ≧900 J/m².

11. The use of a polymerisable mixture composition according to any of claims 1-9 for the production of a plastic mould, characterised in that the polymerised mixture composition or the plastic mould has a fracture toughness (Kₘₐₓ)≧1.9 kJ/m^{1/2} and an energy of rupture 900 J/m².

12. The use according to claim 11, characterised in that the polymerisable mixture composition is used as a repair material for this type of mould or the like.

13. The use of a polymerisable mixture composition according to claim 11 or 12, characterised in that the plastic mould produced is used as a dental prosthesis body and satisfies the minimum requirements according to ISO for high impact plastics.

14. A dental prosthesis, produced from a polymerisable mixture composition according to at least one of claims 1-10.
